(19) United States
(12) Patent Application Publication
(10) Pub. No.: US 2024/0335196 A1
(43) Pub. Date: Oct. 10, 2024

(54) SEALED ELECTRICAL CONNECTION BETWEEN SURGICAL LOADING UNIT AND ELONGATED SHAFT

(71) Applicant: Covidien LP, Mansfield, MA (US)

(72) Inventors: Pawel Abramek, Berlin, CT (US); Kenneth H. Whitfield, North Haven, CT (US); Roanit A. Fernandes, Hamden, CT (US); Kenneth M. Cappola, Monroe, CT (US)

(21) Appl. No.: 18/746,628

(22) Filed: Jun. 18, 2024

Related U.S. Application Data
(63) Continuation of application No. 17/321,855, filed on May 17, 2021, now Pat. No. 12,016,557.
(60) Provisional application No. 63/037,274, filed on Jun. 10, 2020.

(51) Int. Cl. A61B 17/072; A61B 17/00; A61B 17/068; A61B 90/00

(52) U.S. Cl. CPC A61B 17/072; A61B 17/07207; A61B 2017/00017; A61B 2017/00022; A61B 2017/00477; A61B 2017/00486; A61B 17/068; A61B 2017/07228; A61B 2017/07257; A61B 2017/07271; A61B 2090/061

(57) ABSTRACT

A surgical stapling apparatus includes a housing assembly, an elongated shaft extending from the housing assembly, a housing electrical connector assembly, and a loading unit selectively attachable to the elongated shaft. The loading unit extends distally to an end effector supporting at least one sensor. The loading unit supports a loading unit electrical connector assembly on a proximal end portion of loading unit. The loading unit electrical connector assembly is positioned to contact the housing electrical connector assembly when the loading unit electrical connector assembly is received within the elongated shaft to electrically couple the at least one sensor to the housing assembly.

### CROSS REFERENCE TO RELATED APPLICATIONS

This application is a continuation of U.S. patent application Ser. No. 17/321,855, filed May 17, 2021, which claims the benefit of U.S. Provisional patent application Ser. No. 63/037,274, filed Jun. 10, 2020, the entire contents of each of which are hereby incorporated by reference herein.

### TECHNICAL FIELD

This disclosure relates to surgical stapling apparatus and, more particularly, to structures and methods for establishing a sealed electrical connection between a surgical loading unit and an elongated shaft of a powered surgical stapling apparatus.

### BACKGROUND

Fasteners have traditionally been used to replace suturing when joining various body structures. Surgical stapling apparatus employed to apply these fasteners are generally designed to simultaneously cut and seal tissue to reduce the time and risks involved with surgical procedures. Surgical stapling apparatus that clamp, cut and/or staple tissue are well known in the art. Such surgical stapling apparatus include end effectors having two elongated jaw members used to capture or clamp tissue. These end effectors can be provided in the form of an elongate loading unit removably attachable to a housing assembly via an adapter to enable drive components of the housing assembly to operate the end effector in vivo, for instance, laparoscopically. In particular, one of the two jaw members of the end effector usually carries a staple cartridge that houses a plurality of staples positioned in rows, while the other of the two jaw members has an anvil for forming the staples as the staples are driven from the staple cartridge. In linear surgical stapling apparatus, for example, a stapling operation is effectuated by a cam bar, a drive sled or other similar mechanism having a cam member that travels longitudinally through channels defined in the staple cartridge and acts upon staple pushers in the channels to sequentially eject linear rows of staples from the staple cartridge. A knife is movably positioned between the linear rows of staples such that when the surgical stapling apparatus is positioned about tissue and actuated, the tissue is joined and/or simultaneously or nearly simultaneously cut.

### SUMMARY

According to one aspect, a surgical stapling apparatus includes a housing assembly and an elongated shaft assembly. The elongated shaft assembly is selectively attachable to the housing assembly. The elongated shaft assembly includes an adapter assembly and a loading unit. The adapter assembly extends distally to a distal tip housing. The distal tip housing supports an adapter electrical connector assembly therein. The loading unit is selectively attachable to the adapter assembly and extends distally to an end effector supporting one or more sensors therein. The loading unit supports a loading unit electrical connector assembly therein. The loading unit electrical connector assembly is positioned to contact the adapter electrical connector assembly when the adapter assembly and the loading unit are coupled together to electrically couple the one or more sensors to the housing assembly.

In aspects, the one or more sensors may be configured to measure data including thickness of tissue clamped by the end effector, clamp force of the end effector, or firing force of the end effector.

In various aspects, the adapter electrical connector assembly may include an adapter connector housing that rotatably supports a firing rod therethrough. The adapter electrical connector assembly may include an electronic ring assembly that is supported on the adapter connector housing. The adapter connector housing may include a connector shaft that supports the electronic ring assembly thereon. The connector shaft may define a plurality of annular ribs and a plurality of ring recesses disposed between the annular ribs. The plurality of ring recesses and the plurality of annual ribs may be positioned to support a plurality of contact rings of the electronic ring assembly. The plurality of contact rings may be electrically coupled to a flex cable supported by a channel defined within the adapter connector housing. The loading unit electrical connector assembly may include a loading unit connector housing that supports a plurality of spring contacts positioned to contact the plurality of contact rings of the electronic ring assembly. The plurality of spring contacts may be electrically coupled to the one or more sensors.

In aspects, the adapter electrical connector assembly and the loading unit electrical connector assembly may be sealed within elongated shaft assembly when electrically coupled together.

According to yet another aspect, a surgical stapling apparatus includes a housing assembly, an adapter assembly, and a loading unit. The adapter assembly is removably secured to the housing assembly and supports an adapter electrical connector assembly therein. The loading unit is selectively electrically connectable to the adapter assembly by relative translating and rotating movement between the loading unit and the adapter assembly. The loading unit supports a loading unit electrical connector assembly. The loading unit electrical connector assembly is positioned to receive the adapter electrical connector assembly to cause the adapter assembly and the loading unit to electrically couple together in response to the translating and rotating movement.

In aspects, the loading unit may extend to an end effector. The end effector may support one or more sensors disposed in electrical communication with the adapter electrical connector assembly when the loading unit and the adapter assembly are coupled together.

In various aspects, the loading unit may define a lug channel positioned to receive a lug of the adapter assembly. The lug channel may have a longitudinally-extending portion to enable translating movement of the lug therethrough and a transverse portion to enable rotating movement of the lug therethrough.

Other aspects, features, and advantages will be apparent from the description, the drawings, and the claims that follow.

### BRIEF DESCRIPTION OF DRAWINGS

The accompanying drawings, which are incorporated in and constitute a part of this specification, illustrate aspects of the disclosure and, together with a general description of the disclosure given above and the detailed description given below, serve to explain the principles of this disclosure, wherein:

FIG. 1A is a perspective view of an exemplary surgical stapling apparatus in accordance with the principles of this disclosure;

FIG. 1B is a perspective view of an elongated shaft assembly of the surgical stapling apparatus of FIG. 1A, the elongated shaft assembly including a loading unit and an adapter shown coupled together;

FIG. 2 is a perspective view of the elongated shaft assembly of FIG. 1B with the loading unit and the adapter thereof shown separated from one another;

FIG. 3 is an enlarged, perspective view of a proximal end portion of the loading unit and a distal end portion of the adapter, the proximal end portion of the loading unit and the distal end portion of the adapter shown with portions thereof removed and/or in phantom for clarity;

FIG. 4 is an enlarged, perspective view, with parts separated, of a firing rod and an electrical connector assembly of the distal end portion of the adapter;

FIG. 5 is an enlarged, perspective view, with parts separated, of portions of the electrical connector assembly of FIG. 4;

FIGS. 6 and 7 are enlarged, perspective views of the distal end portion of the adapter with portions thereof removed and/or in phantom for clarity;

FIG. 8 is an enlarged, perspective view of the proximal end portion of the loading unit with portions thereof removed and/or in phantom for clarity;

FIG. 9 is an enlarged, perspective view of an electrical connection assembly of the loading unit with portions thereof in phantom for clarity;

FIG. 10 is a perspective view, with parts separated, of the electrical connection assembly of FIG. 9 and a proximal end portion of an outer tube of the loading unit;

FIGS. 11-15 are enlarged, progressive cross-sectional views of the elongated shaft assembly as taken along respective section lines 11-11, 12-12, 13-13, 14-14, and 15-15 shown in FIG. 1B, these views illustrating the loading unit and the adapter being coupled together;

FIG. 16 is a perspective view of a proximal end portion of another loading unit of the of the elongated shaft assembly of FIG. 1B and a distal end portion of another adapter of the elongated shaft assembly of FIG. 1B with portions thereof removed and/or in phantom for clarity;

FIG. 17 is an enlarged, perspective view, in partial cross-section, of the distal end portion of the adapter of FIG. 16;

FIG. 18 is a perspective view of the distal end portion of the adapter of FIG. 16;

FIG. 19 is an enlarged, perspective view of an electrical connector assembly of the adapter of FIG. 16 with portions of the electrical connector assembly in phantom for clarity;

FIG. 20 is another perspective view of the electrical connector assembly of FIG. 19;

FIG. 21 is an enlarged, perspective view, with parts separated, of an electrical connector assembly of the loading unit of FIG. 16;

FIG. 22 is a perspective view of the electrical connector assembly of FIG. 21, with parts assembled;

FIGS. 23-26 are enlarged, progressive cross-sectional views of the adapter and loading unit of FIG. 16 as taken along respective section lines 23-23, 24-24, 25-25 and 26-26 shown in FIG. 27, these views illustrating the loading unit and the adapter of FIG. 16 being coupled together;

FIG. 27 is an enlarged, perspective view illustrating the loading unit and the adapter of FIG. 16 coupled together;

FIG. 28 is an enlarged view of the indicated area of detail shown in FIG. 17 when the adapter and loading unit of FIG. 16 are coupled together as seen in FIG. 27;

FIG. 29 is a perspective view, with parts separated, of yet another proximal end portion of a loading unit and yet another distal end portion of an adapter of the elongated shaft assembly of FIG. 1B;

FIG. 30 is a front view of the proximal end portion of the loading unit of FIG. 29 with portions thereof removed and/or in phantom for clarity;

FIG. 31 is a perspective view of a portion of the adapter of FIG. 29 with portions thereof removed and/or in phantom for clarity;

FIG. 32 is an enlarged, cross-sectional view of portions of the adapter and loading unit of FIG. 29 as taken along section line 32-32 shown in FIG. 31 when the adapter and loading unit are coupled together;

FIG. 33 is a perspective view, with parts separated, of still another proximal end portion of a loading unit of the elongated shaft assembly of FIG. 1B and still another distal end portion of an adapter of the elongated shaft assembly of FIG. 1B with portions thereof removed and/or in phantom for clarity;

FIG. 34 is a perspective view, with parts separated, of an electrical assembly of the adapter of FIG. 33;

FIG. 35 is an enlarged, perspective view illustrating the loading unit and the adapter of FIG. 33 coupled together; and

FIG. 36 is a cross-sectional view of the proximal end portion of the loading unit of FIG. 33 and the distal end portion of the adapter of FIG. 33 coupled together as taken along section line 36-36 shown in FIG. 35.

### DETAILED DESCRIPTION

Aspects of the disclosed surgical stapling apparatus are described in detail with reference to the drawings, in which like reference numerals designate identical or corresponding elements in each of the several views. As commonly known, the term “clinician” refers to a doctor, a nurse, or any other care provider and may include support personnel. Additionally, the term “proximal” refers to the portion of structure that is closer to the clinician and the term “distal” refers to the portion of structure that is farther from the clinician. In addition, directional terms such as front, rear, upper, lower, top, bottom, and the like are used simply for convenience of description and are not intended to limit the disclosure attached hereto.

In the following description, well-known functions or constructions are not described in detail to avoid obscuring the present disclosure in unnecessary detail.

Further, although the surgical instrument described herein is provided in connection with a powered laparoscopic surgical stapling apparatus for brevity, the disclosed surgical instrument can include any powered, manual, or robotically-controlled surgical instruments such as a clip applier, stitching device, energy-based device (e.g., a bipolar or monopolar forceps) or the like, and/or other surgical stapling apparatus such as a circular stapler, a transverse stapler, or an open stapler. For a detailed description of the structure and function of exemplary surgical stapling apparatus, one or more components of which may be included, or modified for use with the disclosed aspects, reference may be made to U.S. Pat. Nos. 9,713,470; 8,806,973; 8,256,656; 8,157,152; 8,070,033 7,819,896; 7,770,774; 7,334,717; 7,128,253; 5,964,394; and 5,915,616, the entire contents of each of which are incorporated herein by reference.

Briefly, due to minerals, ions, etc. in bodily fluids, bodily fluids can be electrically conductive. This disclosure details mechanical structure and methods for securing (and scaling) an electrical connection that resists contamination from body fluids and saline to prevent electronics of the disclosed surgical stapling apparatus from short circuiting. More specifically, this disclosure details structure and methods for effectively relaying information/data (e.g., continuously) from one or more sensors in an end effector of a surgical stapling apparatus at a distal end portion thereof to a housing or handle assembly at a proximal end portion thereof to accurately determine and/or analyze, for example, tissue thickness, clamp force, firing force, etc. using high speed data transfer speeds and a robust sensor signal (e.g., a strain gauge signal).

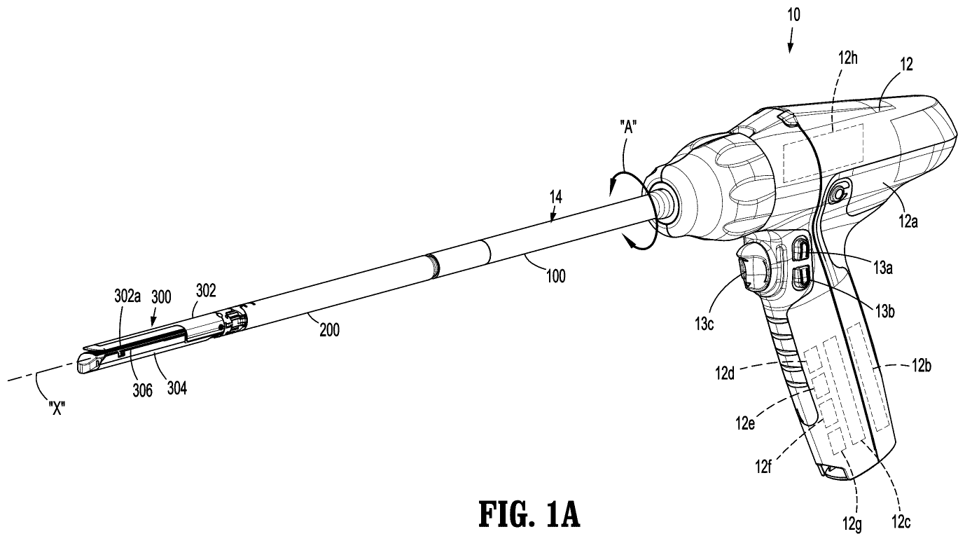

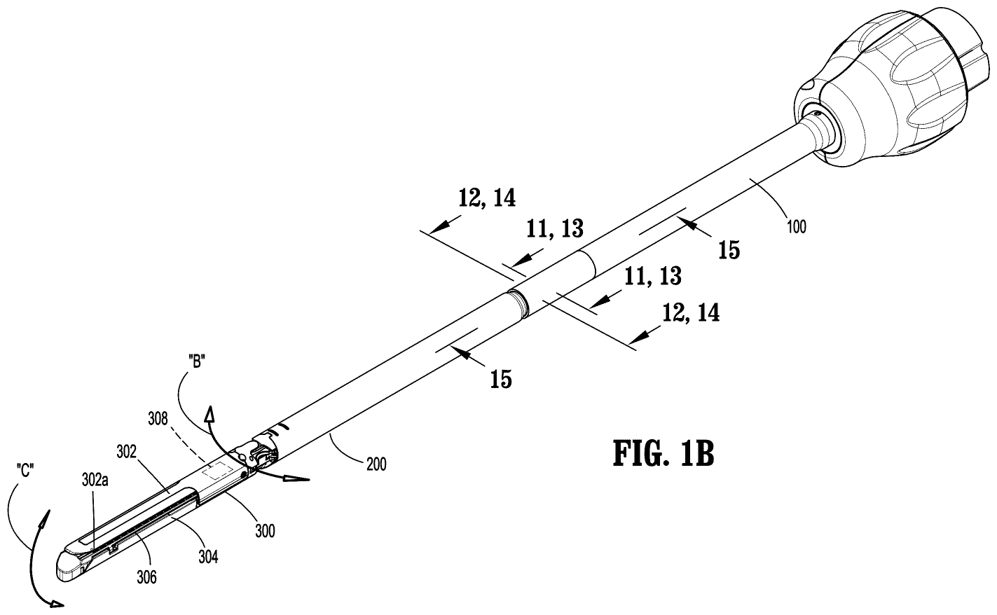

With reference to FIGS. 1A and 1B, a surgical stapling apparatus 10 of this disclosure includes a housing assembly 12 (which may include one or more handles that may be manually actuatable to fire surgical stapling apparatus 10) and an elongated shaft assembly 14 that is removably secured to housing assembly 12. Elongated shaft assembly 14 extends distally to housing assembly 12 and defines a longitudinal axis “X” therealong. Elongated shaft assembly 14 includes an adapter assembly 100 having a proximal end portion removably secured to housing assembly 12. Elongated shaft assembly 14 further includes a loading unit 200 that is removably secured to a distal end portion of adapter assembly 100 and which extends distally from adapter assembly 100 to an end effector 300. Loading unit 200 may be disposable and/or include one or more disposable components. End effector 300 of loading unit 200 includes an anvil assembly 302 and a cartridge assembly 304 that houses a plurality of staples (not shown) in a reload or cartridge 306 thereof that may be selectively replaceable. Anvil assembly 302 includes an anvil 302a against which the plurality of staples is formed upon a firing of surgical stapling apparatus 10. End effector 300 further includes one or more sensors 308 disposed in electrical communication with housing assembly 12. Sensors 308 may include, for example, a strain gauge, a cartridge ID sensor, a near field communications (NFC) antenna, etc. Sensors 308 may be disposed within one or both of anvil assembly 302 and cartridge assembly 304. Sensors 308 are configured to electrically communicate with housing assembly 12 regarding data/information regarding the end effector 300 and/or tissue engaged by end effector 300. For instance, such data/information may relate to tissue thickness, clamp force, firing force, etc.

Housing assembly 12 of surgical stapling apparatus 10 includes a housing 12a configured for selective removable receipt of a rechargeable battery 12b. Battery 12b is configured to supply power to electrical components of surgical stapling apparatus 10. Housing 12a supports a controller 12c (e.g., a circuit board) therein that is configured to control various operations of surgical stapling apparatus 10, and which includes any number of electronic components such as memory 12d, a processor 12e, a network interface 12f, and/or other input/output modules 12g. Controller 12c may be coupled to a local or remote display device (not shown) for outputting information and/or data such as a condition of components of surgical stapling apparatus 10 and/or tissue grasped by end effector 300.

Surgical stapling apparatus 10 further includes a drive mechanism 12h configured to drive mechanical and/or electrical components such as rotatable shafts and/or gear components (not shown) within housing 12a in order to perform various operations of surgical stapling apparatus 10. For instance, drive mechanism 12h may be operable to selectively rotate and/or articulate end effector 300 about, and/or relative to, the longitudinal axis “X” of surgical stapling apparatus 10, as indicated by arrows “A” and “B,” respectively; to selectively move anvil assembly 302 relative to the cartridge assembly 301 and/or vice versa, as indicated by arrows “C” to selectively clamp tissue; and/or to fire surgical stapling apparatus 10 for fastening and/or cutting the clamped tissue. Battery 12b, controller 12c, and/or drive mechanism 12h may be operably coupled to one or more actuators 13a, 13b, and 13c such as finger-actuated control buttons, rocker devices, and/or the like to effectuate various functions of surgical stapling apparatus 10 such as those described above.

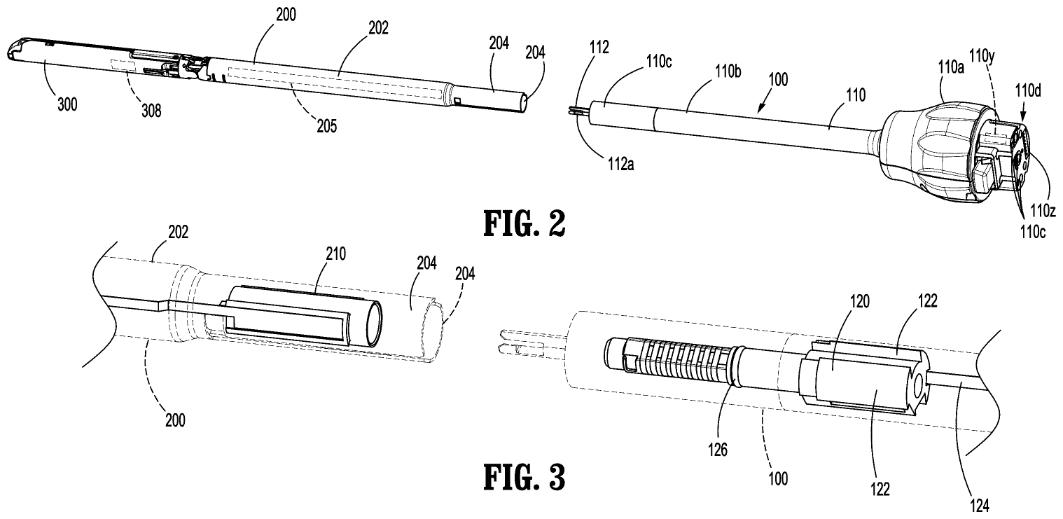

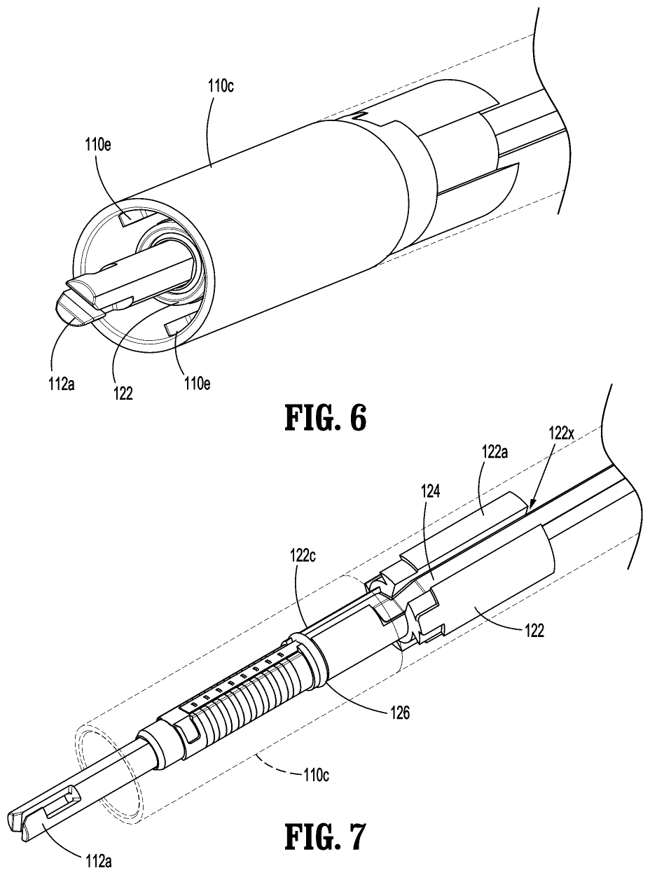

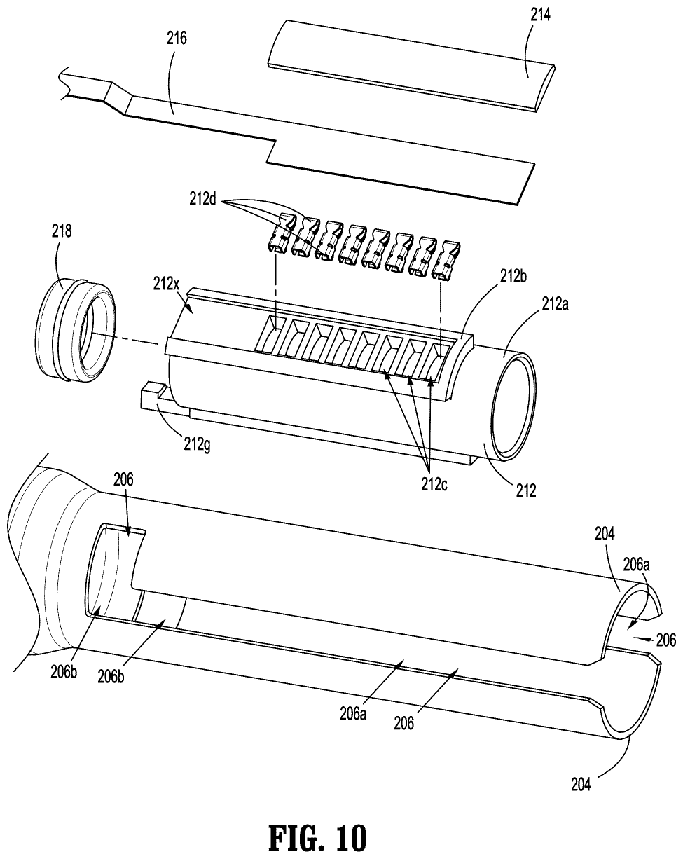

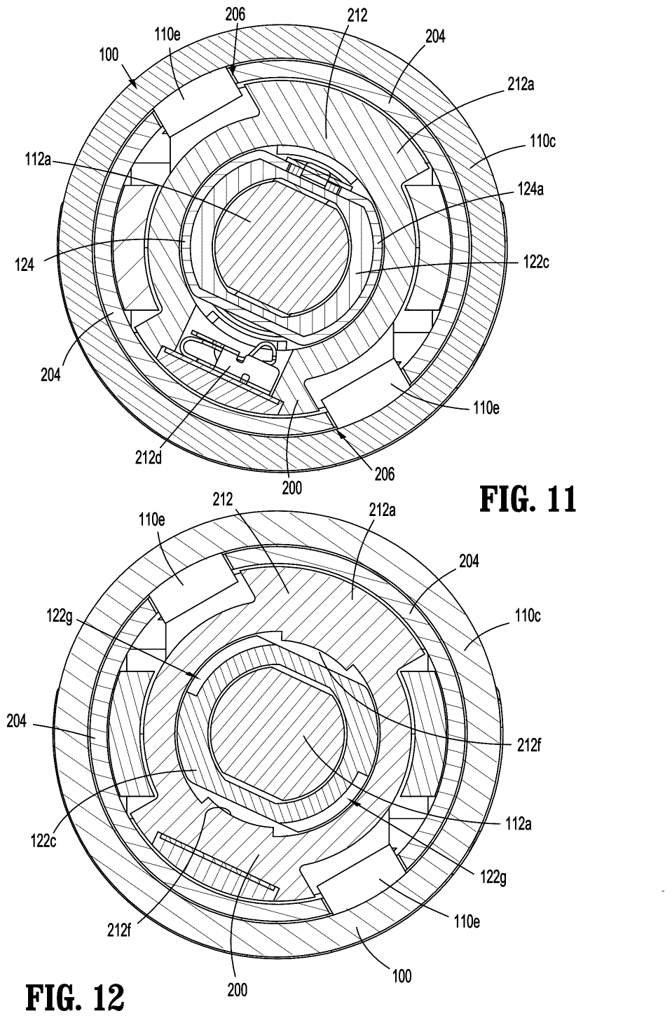

Turning now to FIGS. 2-15, adapter assembly 100 of elongated shaft assembly 14 includes an outer housing 110 and supports a drive assembly 112 therein. Outer housing 110 has a proximal outer housing 110a and a tubular outer housing 110b that extends distally from proximal outer housing 110a to a distal tip housing 110c. Proximal outer housing 110a supports an electrical assembly 110d and a plurality of drive couplers 110c that electromechanically couple to drive mechanism 12h of housing assembly 12. More specifically, electrical assembly 110d includes, for example, an electrical port 110z and a printed circuit board assembly 110y in electrical communication with one another (see FIG. 2). Electrical assembly 110d is configured to electrically communicate with, for example, controller 12c of housing assembly 12 when adapter 100 is coupled to housing assembly 12 while drive couplers 110c mechanically engage drive mechanism 12h, which may include, for instance, a plurality of rotatable actuators (shown) to impart mechanical force (e.g., rotational force) through drive assembly 112 of adapter assembly 100. For example, drive assembly 112 of adapter assembly 100 includes a firing rod 112a that extends distal to distal tip housing 110c and is mechanically engageable with the proximal end portion of loading unit 200 to impart mechanical force (e.g., linear and/or rotational) onto end effector 300 for firing end effector 300 when drive mechanism 12h of housing assembly 12 is actuated. Distal tip housing 110c includes lugs 110e (see FIG. 6) extending radially inward from an inner surface of distal tip housing 110c and positioned to facilitate locking engagement with a proximal end portion of loading unit 200. Lugs 110e may be disposed in diametrically opposed relationship to one another.

Adapter assembly 100 further supports an adapter electrical connector assembly 120 that is disposed in electrical communication with electrical assembly 110d of proximal outer housing 110a. Adapter electrical connector assembly 120 includes an adapter connector housing 122 that is positioned to receive firing rod 112a therethrough so that firing rod 112a is rotatable relative to adapter connector housing 122. Adapter electrical connector assembly 120 further includes an electronic ring assembly 124 and a seal 126 (e.g., an annular seal or gasket such as an O-ring) that are secured to adapter connector housing 122.

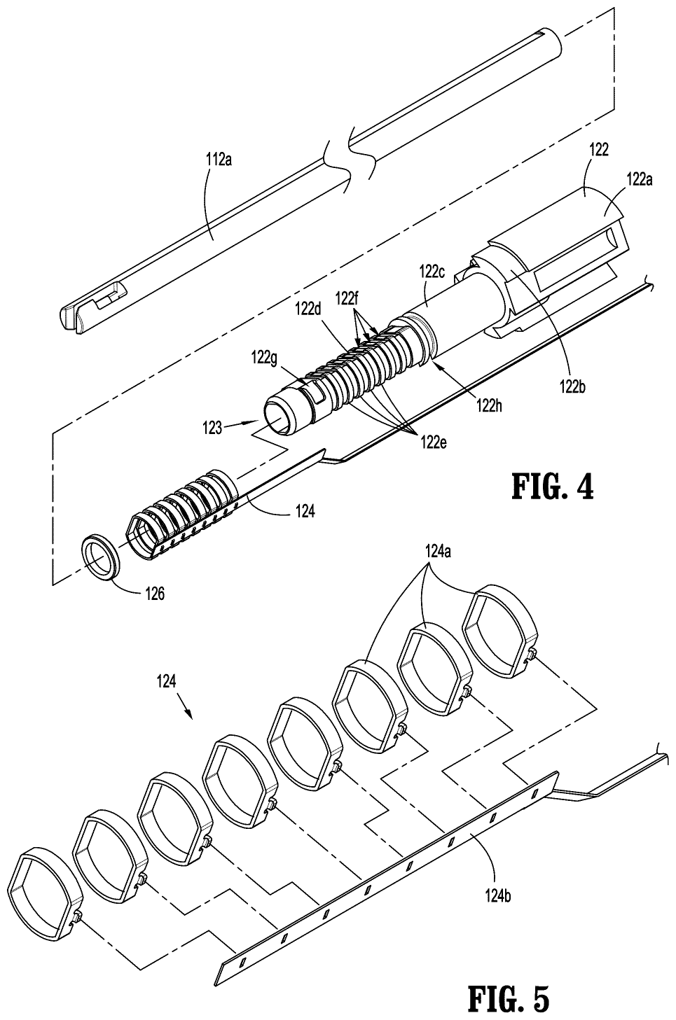

As best seen in FIGS. 2-7, adapter connector housing 122 of adapter electrical connector assembly 120 is supported within tubular outer housing 110b of adapter assembly 100. Adapter connector housing 122 includes a proximal base 122a having a distal ledge 122b recessed from proximal base 122a to enable adapter connector housing 122 to couple to a proximal end portion of distal tip housing 110c of adapter assembly 100. Adapter connector housing 122, which may be wholly or partially non-conductive, further includes a connector shaft 122c that extends distally from proximal base 122a for supporting electronic ring assembly 124 and seal 126. Connector shaft 122c and proximal base 122a define a flex channel 122x (see FIG. 7) along an outer surface thereof for supporting electronic ring assembly 124 and a central lumen 123 therethrough for rotatably receiving firing rod 112a therethrough. Connector shaft 122c includes a mounting finger 122d having a plurality of annular ribs 122e that are longitudinally spaced apart along an outer surface of mounting finger 122d to define ring recesses 122f between adjacent annular ribs 122e for receiving electronic ring assembly 124. Connector shaft 122c further defines a pair alignment notches 122g disposed in diametrical opposed relation to one another (see FIGS. 4 and 12) on the outer surface of connector shaft 122c and distal to the plurality of annular ribs 122c to facilitate engagement with loading unit 200 (and to help maintain proper positioning of ribs 122e for isolating electrical contacts). Connector shaft 122c further defines an annular seal channel 122h for mounting seal 126 to adapter connector housing 122 over electronic ring assembly 124 (e.g., overmolded or assembled). Electronic ring assembly 124 includes a plurality of longitudinally spaced apart contact rings 124a, which are conductive (e.g., metallic), that are secured within ring recesses 122f of mounting finger 122d (e.g., insert molded) and are coupled to a connector flex assembly 124b (e.g., soldered) that is supported within flex channel 122x of adapter connector housing 122. Connector flex assembly 124b, which may be in the form of a flex cable for electrically communicating data and/or power, extends proximally from adapter connector housing 122 and is disposed in electrical communication with electrical assembly 110d of adapter assembly 100.

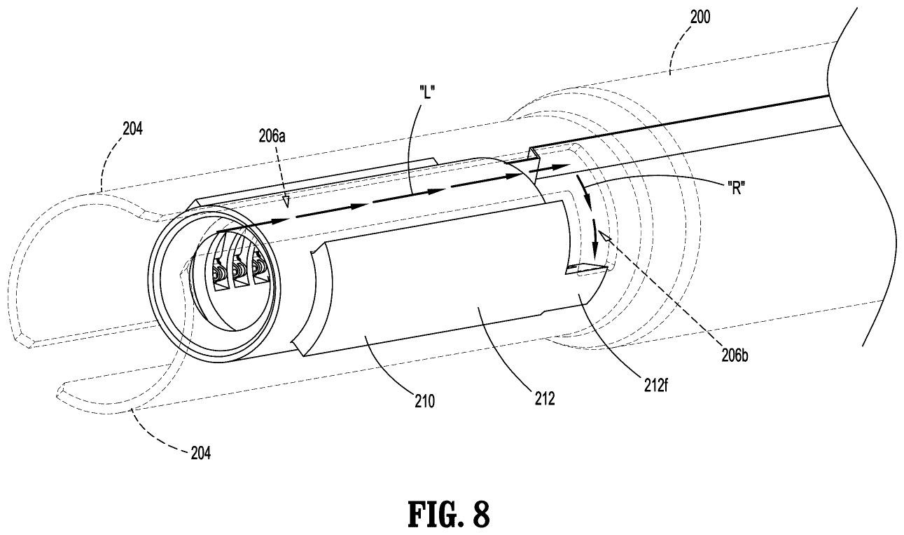

With reference to FIGS. 2, 3, and 8-11, loading unit 200 of elongated shaft assembly 14 has a tubular shaft 202 that supports a loading unit drive assembly 205 therein that is configured to couple to drive assembly 112 of adapter assembly 100 to operate end effector 300. A proximal portion of tubular shaft 202 of loading unit 200 has a pair of curved tines 204 disposed in mirrored relationship with one another (e.g., diametrically opposed) and which extend to a proximal end of loading unit 200. Tines 204 of tubular shaft 202 are receivable within distal tip housing 110c of adapter assembly 100. The curved tines 204 define a pair of outer lug channels 206 for receiving lugs 110e of adapter assembly 100 (see FIG. 6) therein to secure loading unit 200 and adapter assembly 100 together. Outer lug channels 206 of loading unit 200 include a longitudinally-extending portion 206a for longitudinally receiving lugs 110e, as indicated by arrows “L” in FIG. 8, and a transverse portion 206b at a distal end of longitudinally-extending portion 206a for rotatably receiving lugs 110e therein, as indicated by arrows “R” in FIG. 8, to lock loading unit 200 and adapter assembly 100 together.

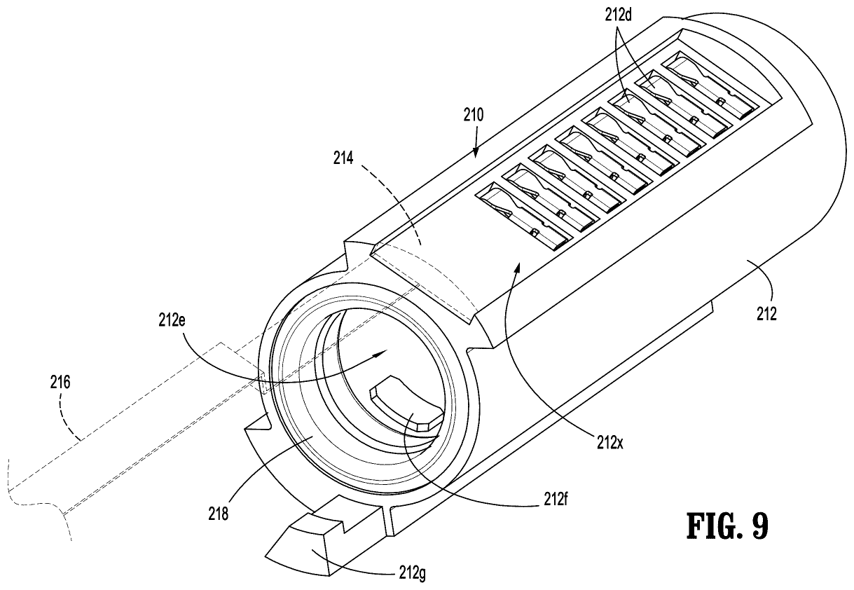

Loading unit 200 of elongated shaft assembly 14 supports a loading unit electrical connector assembly 210 between the pair of curved tines 204. Loading unit electrical connector assembly 210 extends distally through tubular shaft 202 for electrically coupling to sensors 308 supported within end effector 300, and 210 includes a loading unit connector housing 212 (wholly or partially non-conductive) having a tubular body 212a that supports an outer rail 212b. Outer rail 212b defines a series of spring contact recesses 212c therein. The spring contact recesses 212c are longitudinally spaced apart from one another. Spring contact recesses 212c support a series of spring contacts 212d, which are electrically conductive (e.g., metallic). Outer rail 212b further defines rail channel 212x therein that extends longitudinally along outer rail 212b. Tubular body 212a defines a central passage 212e therethrough and which is configured to receive adapter electrical connector assembly 120 of adapter assembly 100 therein and firing rod 112a of adapter assembly 100 therethrough. Tubular body 212a further includes a pair of tabs 212f (see FIGS. 9 and 12) extending radially inward from an inner surface of tubular body 212a. Tabs 212f are positioned to engage the pair alignment notches 122g defined in connector shaft 122c of adapter connector housing 122 (see FIG. 4) to facilitate securement of loading unit 200 and adapter assembly 100 together. Tubular body 212a further includes a distal tooth 212g which functions as a rotational stop for lugs 110e of adapter assembly 100 (see FIG. 8) and a retention feature that keeps loading unit connector housing 212 engaged with loading unit 200. Loading unit electrical connector assembly 210 further includes a seal cap 214, a loading unit flex assembly 216, which may be in the form of a flex cable, and a seal ring 218 (e.g., an O-ring). Seal cap 214 is mounted in rail channel 212x of outer rail 212b over a backside of loading unit flex assembly 216 and is configured to secure spring contacts 212d within outer rail 212b and to stiffen and seal the backside of loading unit flex assembly 216. Loading unit flex assembly 216 extends distally through loading unit 200 to electrically couple to sensors 308 within end effector 300. Seal ring 218 seats in a distal portion of central passage 212e of tubular body 212a of loading unit electrical connector assembly 210 to seal the central passage 212e of tubular body 212a.

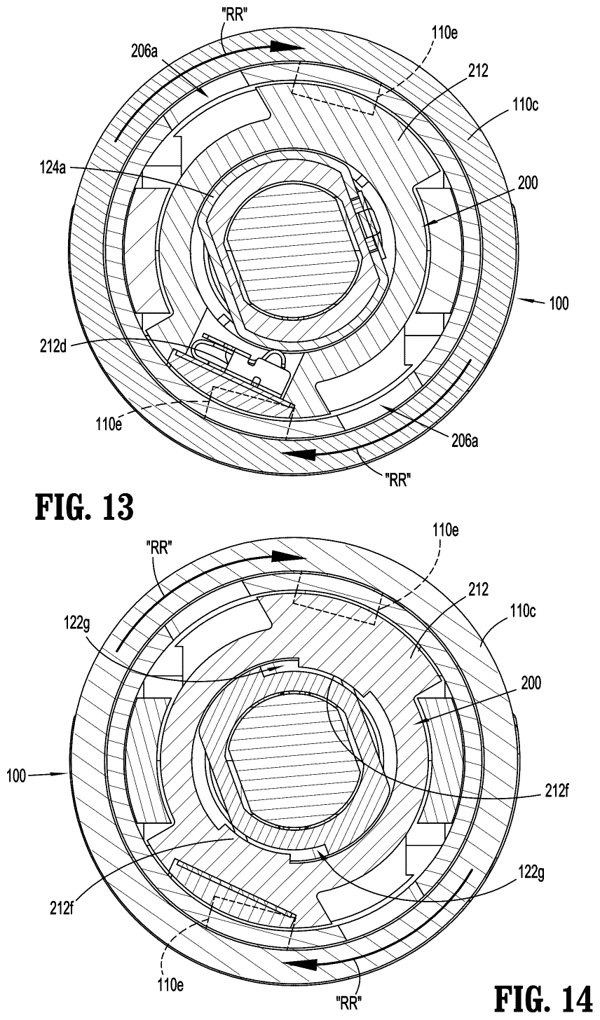

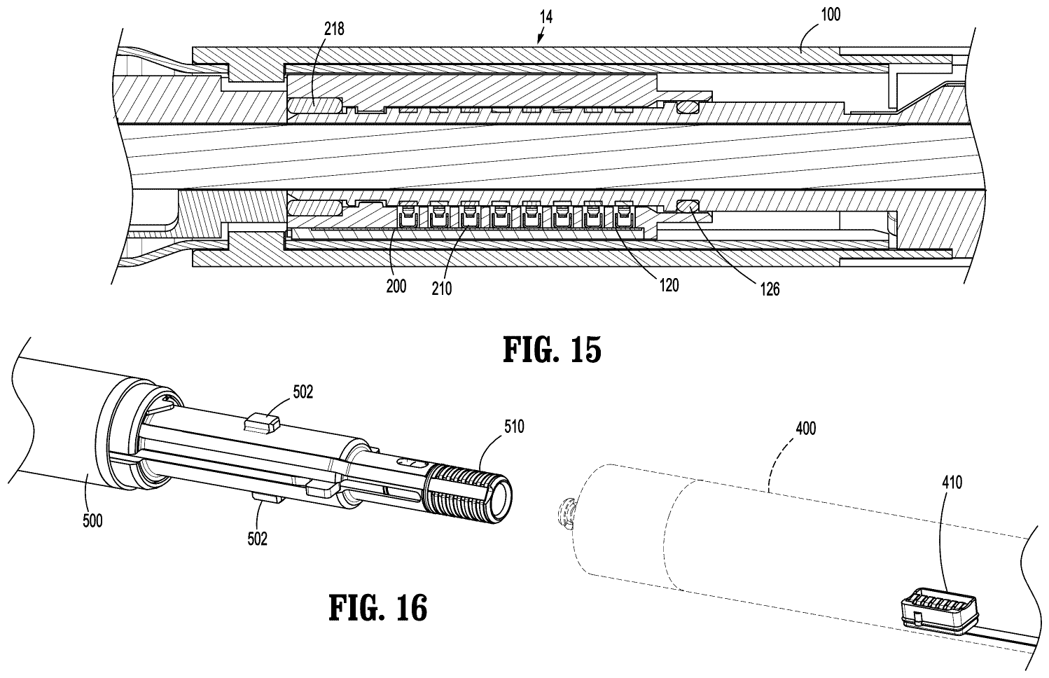

With reference to FIGS. 8 and 11-15, to mechanical and electrically couple adapter assembly 100 and loading unit 200 together, the curved tines 204 of tubular shaft 202 of loading unit 200 is inserted within distal tip housing 110c of adapter assembly 100 so that lugs 110c of distal tip housing 110c translate distally along outer lug channels 206. Lugs 110e are advanced distally along longitudinally-extending portion 206a of outer lug channel 206, as indicated by arrows “L” (see FIG. 8) until longitudinally aligned with transverse portion 206b of outer lug channel 206, and tabs 212f of tubular body 212a are longitudinally aligned with alignment notches 122g of connector shaft 122c. Then, relative rotation between adapter assembly 100 and loading unit 200, as indicated by arrows “RR” shown in FIGS. 13 and 14, causes lugs 110e to rotate into transverse portion 206b of outer lug channel 206, as indicated by arrows “R” in FIG. 8 and tabs 212f to rotate into alignment notches 122g. In this position, adapter assembly 100 and loading unit 200 are mechanical locked together and electrically coupled together via contact between adapter electrical connector assembly 120 and loading unit electrical connector assembly 210 as seen in FIG. 15 so that an electrical circuit is formed from sensors 308 in end effector 300 through elongated shaft assembly 14, and to housing assembly 12 (e.g., controller 12c, battery 12b, etc., thereof.) In this position, adapter electrical connector assembly 120 and loading unit electrical assembly 210 are sealed via seal ring 218 and seal 126.

Once the electrical circuit is created, surgical stapling apparatus 10 can be used to effectuate a surgical procedure, whereby the electrical circuit can determine and/or analyze data/information may relate to tissue thickness, clamp force, firing force, etc. to help facilitate the efficiency and effectiveness of the surgical procedure. Loading unit 200 can be separated and removed from adapter assembly 100 as desired, for example, to dispose of and/or replace the loading unit 200 with another loading unit 200. Adapter assembly 100 is likewise removable and replaceable with respect to loading unit 200 and/or housing assembly 12.

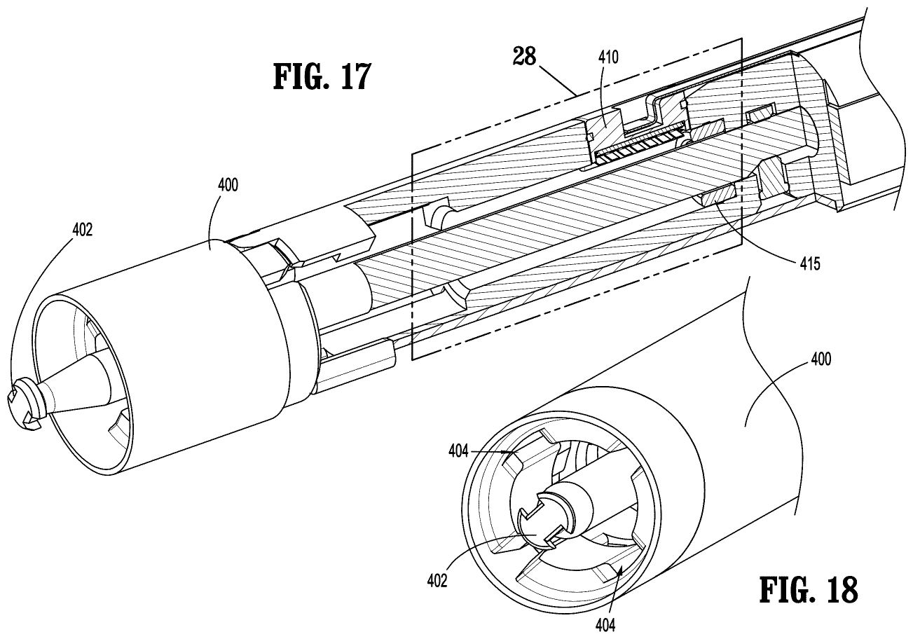

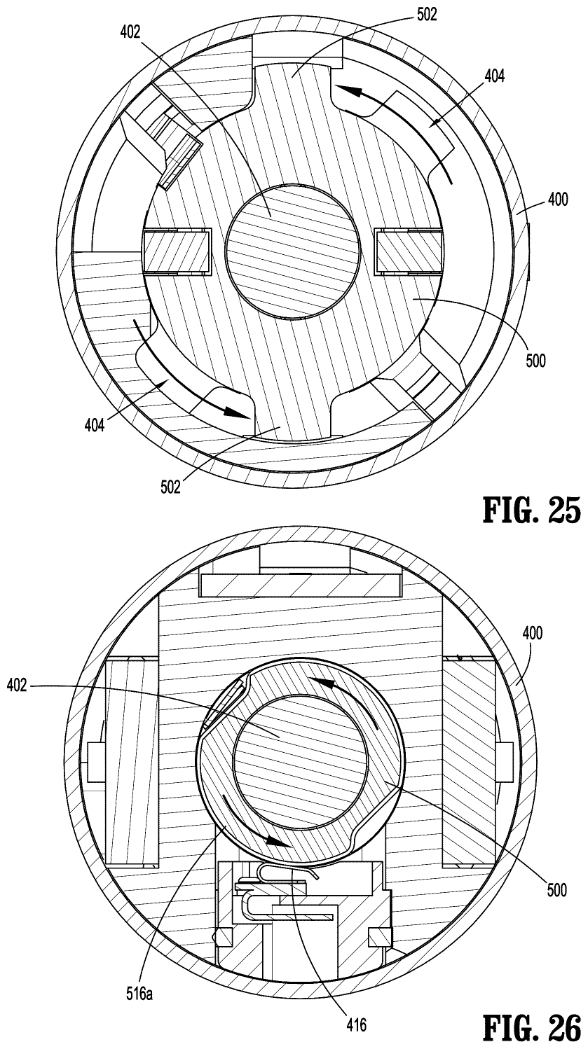

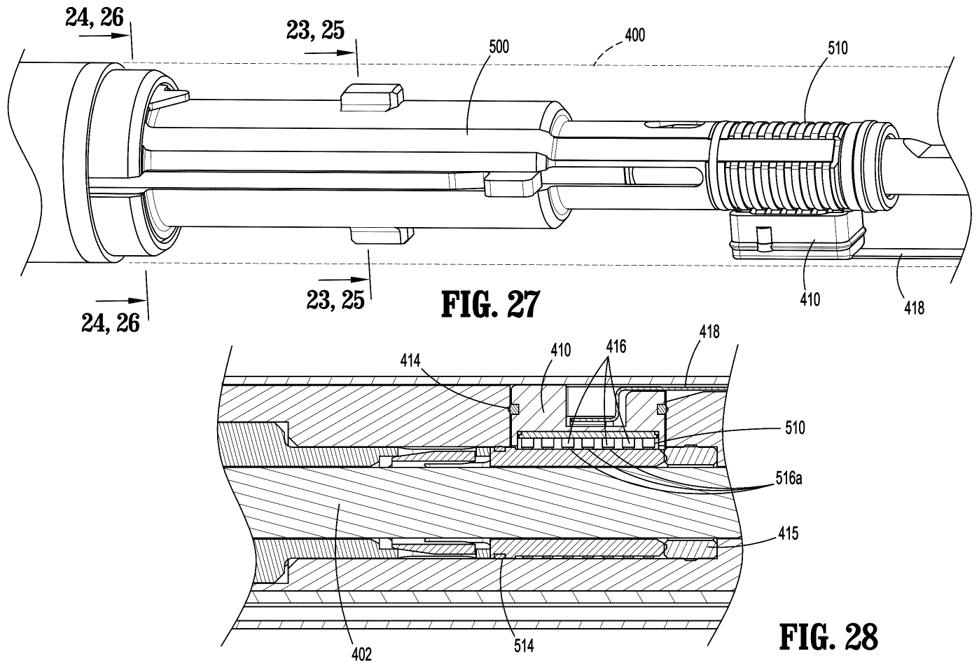

Turning now to FIGS. 16-28, according to another aspect, adapter assembly 400 and a loading unit 500 can also be removably, electromechanically coupled together similar to adapter 100 and loading unit 200. Adapter assembly 400 includes adapter electrical connector assembly 410 and loading unit 500 includes loading unit electrical connector assembly 510. Adapter electrical connector assembly 410 of adapter assembly 400 couples to electrical assembly 110d at the proximal end portion of adapter assembly 400 and loading unit electrical connector assembly 510 couples to sensors 308 supported in end effector 300. Adapter assembly 400 supports a firing rod 402 and defines lug slots 404 therein for receiving lugs 502 extending radially outward from the proximal end portion of loading unit 500. Adapter assembly 400 further includes seal ring 415 supported about firing rod 402 proximal to adapter electrical connector assembly 410.

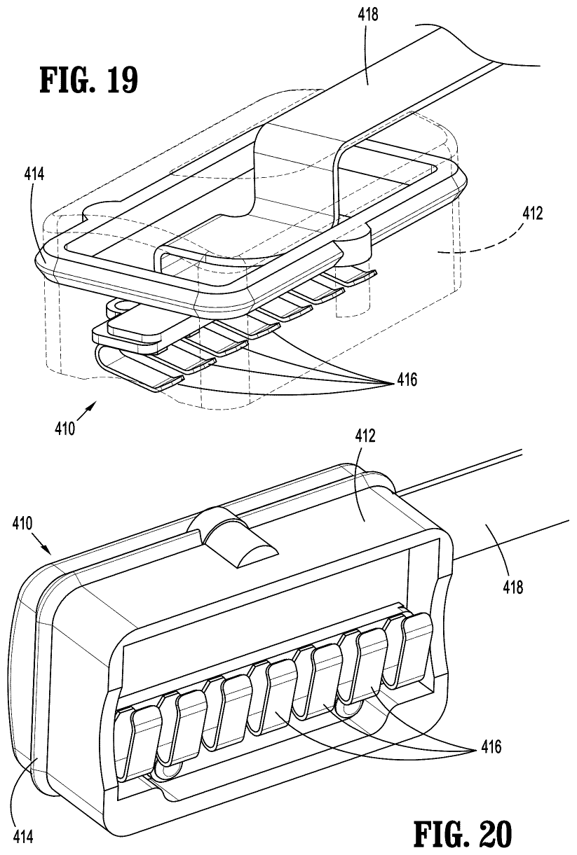

As best seen in FIGS. 19 and 20, adapter electrical connector assembly 410 includes a connector housing 412 and a peripheral seal 414 secured onto connector housing 412 (e.g., overmolded). Adapter electrical connector assembly 410 further includes a plurality of spring contacts 416, which are electrically conductive, supported in connector housing 412 and longitudinally spaced apart from one another. Spring contacts 416 are coupled to a flex cable 418 (e.g., soldered thereto).

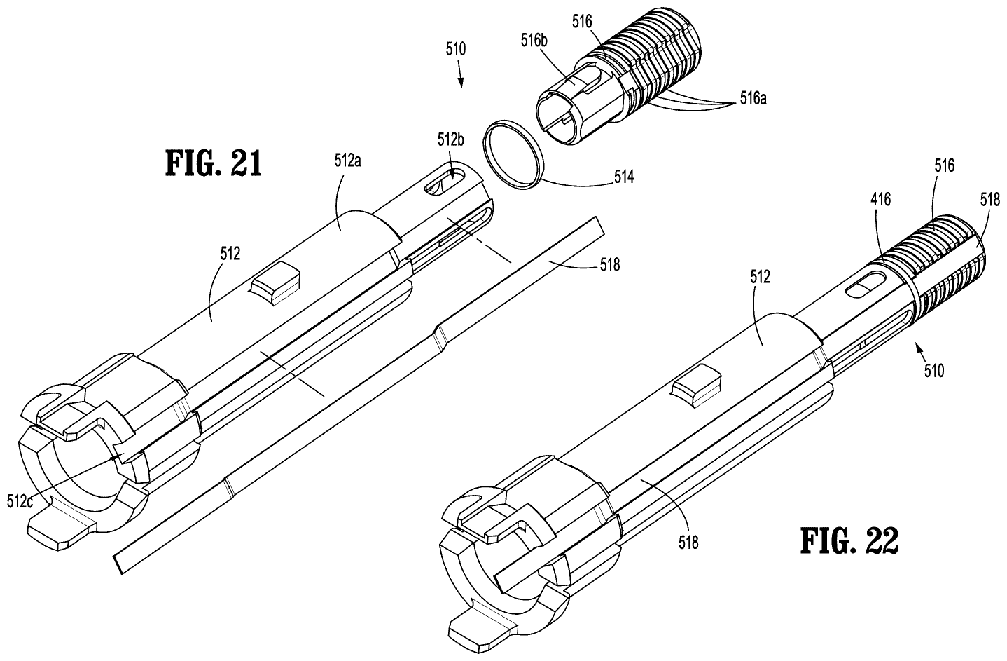

With reference to FIGS. 21 and 22, loading unit electrical connector assembly 510 includes a connector housing 512 that has a tubular body 512a. Tubular body 512 defines snap-fit apertures 512b through a sidewall of tubular body 512a. Tubular body 512 further defines a cable channel 512c along an outer surface of tubular body 512a. Loading unit electrical connector assembly 510 further includes a seal 514 (e.g., an O-ring), an electrical coupler 516 onto which seal 514 mounts, and a flex cable 518. Electrical coupler 516 includes a plurality of longitudinally spaced apart contract rings 516a, each of which is electrically conductive, and a pair of snap-fit arms 516b flexibly mounted thereto. Snap-fit arms 516b are configured to snap-fit into snap-fit apertures 512b of tubular body 512 to secure electrical coupler 516 to tubular body 512 as seen in FIG. 22.

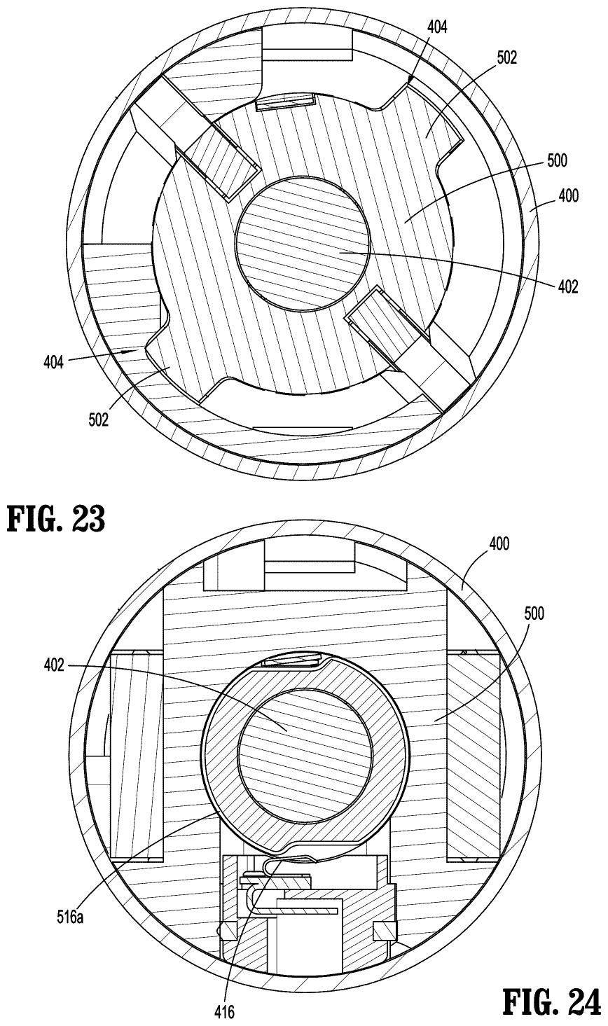

With reference to FIGS. 23-28, to electromechanically couple loading unit 500 to adapter assembly 400, loading unit 500 is axially inserted into adapter assembly 400 and rotated similar to loading unit 200 and adapter assembly 400, as detailed above, so that loading unit electrical connector assembly 510 and adapter electrical connector assembly 410 electrically couple together.

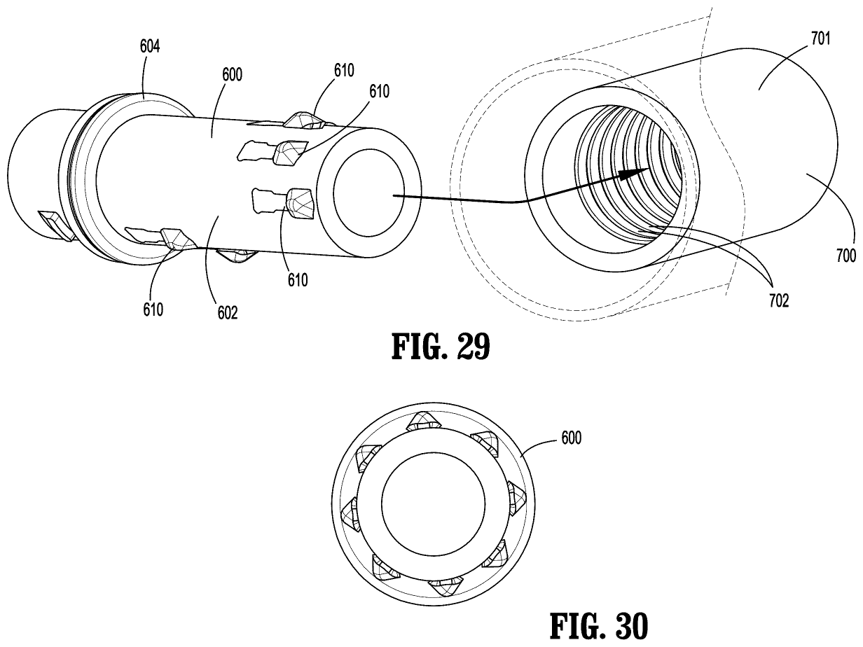

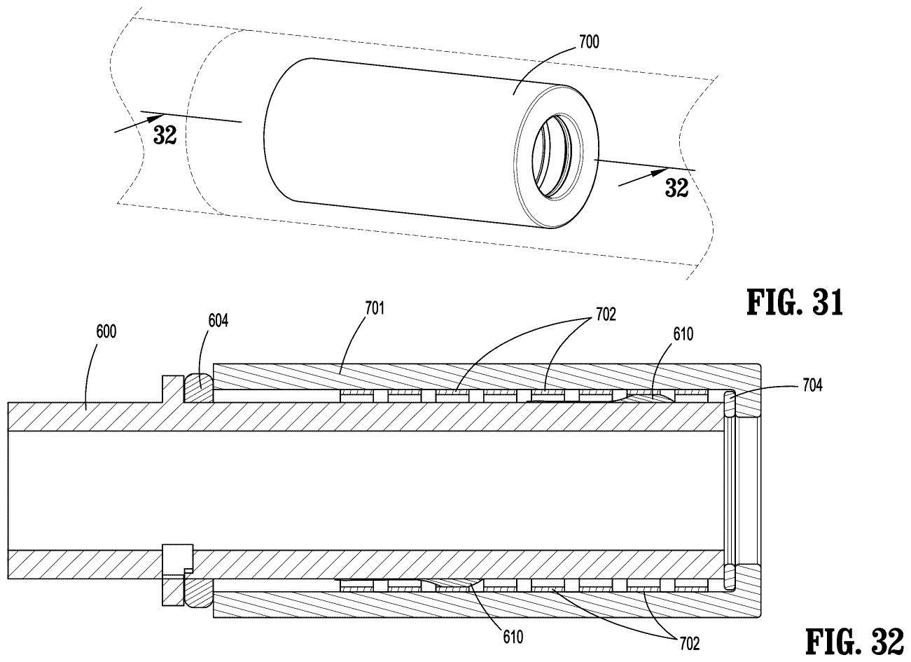

Turning now to FIGS. 29-32, according to yet another aspect, a loading unit electrical connector assembly 600 can be electrically coupled to an adapter electrical connector assembly 700. Loading unit electrical connector assembly 600 includes a plurality of sheet metal contacts 610, each of which is electrically conductive, angularly spaced about a tubular body 602 of loading unit electrical connector assembly 600 and seal 604 supported on tubular body 602. Sheet metal contacts 610 may be angularly and/or longitudinally spaced apart from one another. In aspects, sheet metal contacts 610 may be disposed in a spiral arrangement about tubular body 602. Adapter electrical connector assembly 700 includes a plurality of annular contact rings 702, each of which is electrically conductive. The annular contact rings 702 are longitudinally spaced apart along an inner surface of a tubular body 701 of adapter electrical connector assembly 700. Adapter electrical connector assembly 700 further includes a seal 704 supported therein.

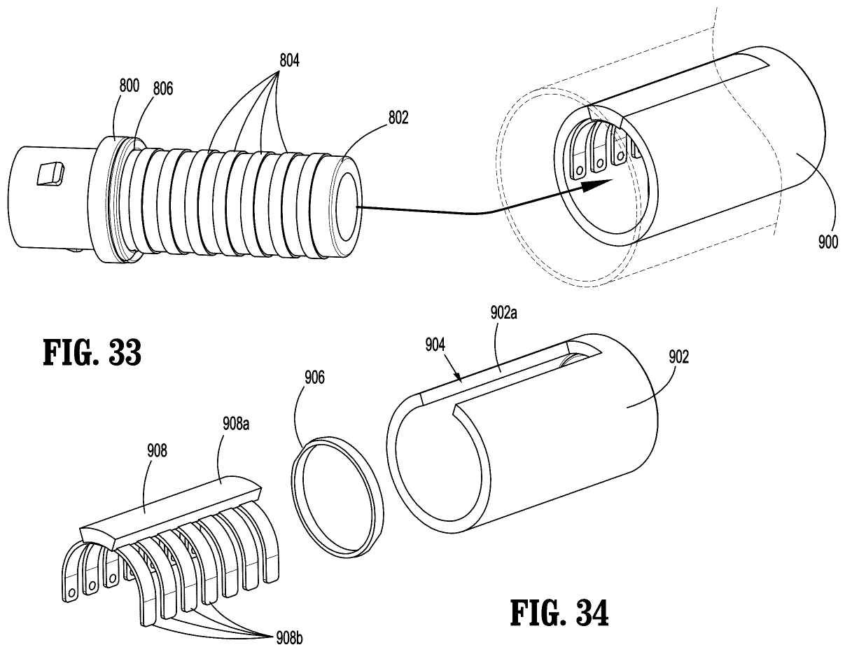

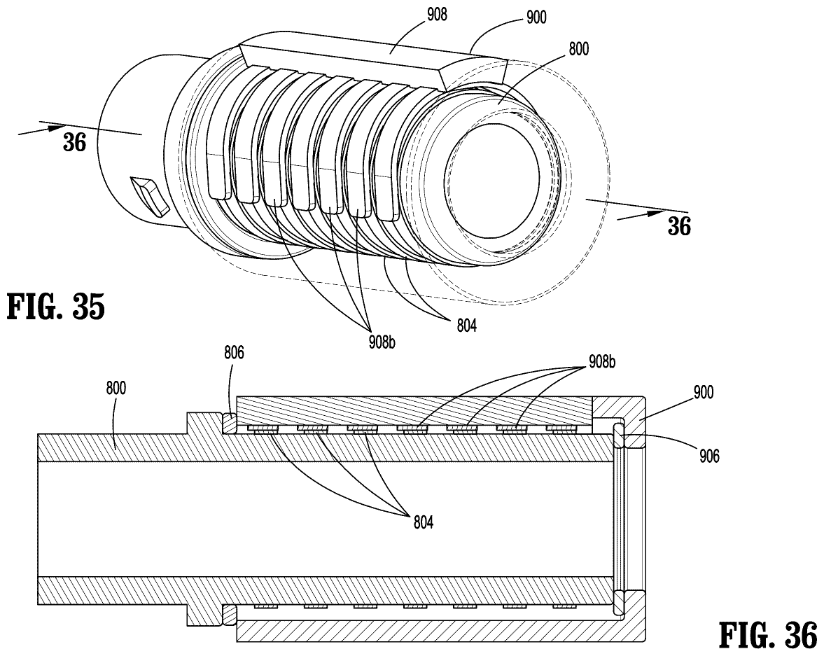

With reference to FIGS. 33-36, according to still another aspect, a loading unit electrical connector assembly 800 can be electrically coupled to an adapter electrical connector assembly 900. Loading unit electrical connector assembly 800 includes a tubular body 802 supporting a plurality of contact rings 804, each of which is electrically conductive, at longitudinally spaced apart locations and a seal 806. Adapter electrical connector assembly 900 includes a tubular body 902 defining a cutout 904 that extends longitudinally along sidewall 902a of tubular body 902. Adapter electrical connector assembly 900 further includes a seal 906 and a contact insert assembly 908 that is receivable in cutout 907 of tubular body 902. Contact insert assembly 908 includes an elongate leg 908a and a plurality of arched contacts 908b, each of which is electrically conductive, longitudinally spaced apart along elongate leg 908a and receivable within tubular body 902 when elongate leg 908a is seated in cutout 904 of tubular body 902.

Further, the various aspects disclosed herein may also be configured to work with robotic surgical systems and what is commonly referred to as “Telesurgery.” Such systems employ various robotic elements to assist the clinician and allow remote operation (or partial remote operation) of surgical instrumentation. Various robotic arms, gears, cams, pulleys, electric and mechanical motors, etc. may be employed for this purpose and may be designed with a robotic surgical system to assist the clinician during the course of an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of clinicians may prep the patient for surgery and configure the robotic surgical system with one or more of the instruments disclosed herein while another clinician (or group of clinicians) remotely controls the instruments via the robotic surgical system. As can be appreciated, a highly skilled clinician may perform multiple operations in multiple locations without leaving his/her remote console which can be both economically advantageous and a benefit to the patient or a series of patients. For a detailed description of exemplary medical work stations and/or components thereof, reference may be made to U.S. Patent Application Publication No. 2012/0116416, and PCT application Publication No. WO2016/025132, the entire contents of each of which are incorporated by reference herein.

Moreover, the disclosed electronic structure such as the electronic assembly and/or controllers, can include any suitable electrical components for operating the disclosed surgical stapling apparatus or components thereof. Such electrical components can include, for example, one or more controllers and/or circuitry, which may include or be coupled to one or more printed circuit boards. As used herein, the term “controller” includes “processor,” “digital processing device” and like terms, and are used to indicate a microprocessor or central processing unit (CPU). The CPU is the electronic circuitry within a computer that carries out the instructions of a computer program by performing the basic arithmetic, logical, control and input/output (I/O) operations specified by the instructions, and by way of non-limiting examples, include server computers. In some aspects, the controller includes an operating system configured to perform executable instructions. The operating system is, for example, software, including programs and data, which manages hardware of the disclosed surgical stapling apparatus and provides services for execution of applications for use with the disclosed surgical stapling apparatus. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. In some aspects, the operating system is provided by cloud computing.

In some aspects, the term “controller” may be used to indicate a device that controls the transfer of data from a computer or computing device to a peripheral or separate device and vice versa, and/or a mechanical and/or electromechanical device (e.g., a lever, knob, etc.) that mechanically operates and/or actuates a peripheral or separate device.

In aspects, the controller includes a storage and/or memory device. The storage and/or memory device is one or more physical apparatus used to store data or programs on a temporary or permanent basis. In some aspects, the controller includes volatile memory and requires power to maintain stored information. In various aspects, the controller includes non-volatile memory and retains stored information when it is not powered. In some aspects, the non-volatile memory includes flash memory. In certain aspects, the non-volatile memory includes dynamic random-access memory (DRAM). In some aspects, the non-volatile memory includes ferroelectric random access memory (FRAM). In various aspects, the non-volatile memory includes phase-change random access memory (PRAM). In certain aspects, the controller is a storage device including, by way of non-limiting examples, CD-ROMs, DVDs, flash memory devices, magnetic disk drives, magnetic tapes drives, optical disk drives, and cloud computing based storage. In various aspects, the storage and/or memory device is a combination of devices such as those disclosed herein.

In some aspects, the controller includes a display to send visual information to a user. In various aspects, the display is a cathode ray tube (CRT). In various aspects, the display is a liquid crystal display (LCD). In certain aspects, the display is a thin film transistor liquid crystal display (TFT-LCD). In aspects, the display is an organic light emitting diode (OLED) display. In certain aspects, on OLED display is a passive-matrix OLED (PMOLED) or active-matrix OLED (AMOLED) display. In aspects, the display is a plasma display. In certain aspects, the display is a video projector. In various aspects, the display is interactive (e.g., having a touch screen or a sensor such as a camera, a 3D sensor, a LiDAR, a radar, etc.) that can detect user interactions/gestures/responses and the like. In some aspects, the display is a combination of devices such as those disclosed herein.

The controller may include or be coupled to a server and/or a network. As used herein, the term “server” includes “computer server,” “central server,” “main server,” and like terms to indicate a computer or device on a network that manages the surgical stapling apparatus, components thereof, and/or resources thereof. As used herein, the term “network” can include any network technology including, for instance, a cellular data network, a wired network, a fiber optic network, a satellite network, and/or an IEEE 802.11a/b/g/n/ac wireless network, among others.

In various aspects, the controller can be coupled to a mesh network. As used herein, a “mesh network” is a network topology in which each node relays data for the network. All mesh nodes cooperate in the distribution of data in the network. It can be applied to both wired and wireless networks. Wireless mesh networks can be considered a type of “Wireless ad hoc” network. Thus, wireless mesh networks are closely related to Mobile ad hoc networks (MANETs). Although MANETs are not restricted to a specific mesh network topology, Wireless ad hoc networks or MANETs can take any form of network topology. Mesh networks can relay messages using either a flooding technique or a routing technique. With routing, the message is propagated along a path by hopping from node to node until it reaches its destination. To ensure that all its paths are available, the network must allow for continuous connections and must reconfigure itself around broken paths, using self-healing algorithms such as Shortest Path Bridging. Self-healing allows a routing-based network to operate when a node breaks down or when a connection becomes unreliable. As a result, the network is typically quite reliable, as there is often more than one path between a source and a destination in the network. This concept can also apply to wired networks and to software interaction. A mesh network whose nodes are all connected to each other is a fully connected network.

In some aspects, the controller may include one or more modules. As used herein, the term “module” and like terms are used to indicate a self-contained hardware component of the central server, which in turn includes software modules. In software, a module is a part of a program. Programs are composed of one or more independently developed modules that are not combined until the program is linked. A single module can contain one or several routines, or sections of programs that perform a particular task.

As used herein, the controller includes software modules for managing various aspects and functions of the disclosed surgical stapling apparatus or components thereof.

The disclosed surgical stapling apparatus may also utilize one or more controllers to receive various information and transform the received information to generate an output. The controller may include any type of computing device, computational circuit, or any type of processor or processing circuit capable of executing a series of instructions that are stored in memory. The controller may include multiple processors and/or multicore central processing units (CPUs) and may include any type of processor, such as a microprocessor, digital signal processor, microcontroller, programmable logic device (PLD), field programmable gate array (FPGA), or the like. The controller may also include a memory to store data and/or instructions that, when executed by the one or more processors, cause the one or more processors to perform one or more methods and/or algorithms.

Any of the herein described methods, programs, algorithms or codes may be converted to, or expressed in, a programming language or computer program. The terms “programming language” and “computer program,” as used herein, each include any language used to specify instructions to a computer, and include (but is not limited to) the following languages and their derivatives: Assembler, Basic, Batch files, BCPL, C, C+, C++, Delphi, Fortran, Java, JavaScript, machine code, operating system command languages, Pascal, Perl, PL1, scripting languages, Visual Basic, metalanguages which themselves specify programs, and all first, second, third, fourth, fifth, or further generation computer languages. Also included are database and other data schemas, and any other meta-languages. No distinction is made between languages which are interpreted, compiled, or use both compiled and interpreted approaches. No distinction is made between compiled and source versions of a program. Thus, reference to a program, where the programming language could exist in more than one state (such as source, compiled, object, or linked) is a reference to any and all such states. Reference to a program may encompass the actual instructions and/or the intent of those instructions.

As can be appreciated, securement of any of the components of the disclosed apparatus can be effectuated using known securement techniques such welding, crimping, gluing, fastening, etc. Also, any of the disclosed structure can include any suitable conductive material (e.g., metallic), semi-conductive material (e.g., silicone), and/or non-conductive/insulative material (e.g., plastic).

Persons skilled in the art will understand that the structures and methods specifically described herein and illustrated in the accompanying figures are non-limiting exemplary aspects, and that the description, disclosure, and figures should be construed merely as exemplary of particular aspects. It is to be understood, therefore, that this disclosure is not limited to the precise aspects described, and that various other changes and modifications may be effectuated by one skilled in the art without departing from the scope or spirit of the disclosure. Additionally, it is envisioned that the elements and features illustrated or described in connection with one exemplary aspect may be combined with the elements and features of another without departing from the scope of this disclosure, and that such modifications and variations are also intended to be included within the scope of this disclosure. Indeed, any combination of any of the disclosed elements and features is within the scope of this disclosure. Accordingly, the subject matter of this disclosure is not to be limited by what has been particularly shown and described.

1-20. (canceled)

21. A surgical stapling apparatus, comprising:
a housing assembly; and
an elongated shaft extending from the housing assembly;
a housing electrical connector assembly; and
a loading unit selectively attachable to the elongated shaft and extending distally to an end effector supporting at least one sensor, the loading unit supporting a loading unit electrical connector assembly on a proximal end portion of loading unit, the loading unit electrical connector assembly positioned to contact the housing electrical connector assembly when the loading unit electrical connector assembly is received within the elongated shaft to electrically couple the at least one sensor to the housing assembly.

22. The surgical stapling apparatus of claim 21, wherein the at least one sensor is configured to measure data including thickness of tissue clamped by the end effector, clamp force of the end effector, or firing force of the end effector.

23. The surgical stapling apparatus of claim 22, wherein the housing electrical connector assembly couples to an electrical assembly at a proximal end portion of the elongated shaft.

24. The surgical stapling apparatus of claim 23, wherein the loading unit electrical connector assembly is electrically coupled to the at least one sensor.

25. The surgical stapling apparatus of claim 24, wherein the elongated shaft supports a firing rod and defines lug slots therein for receiving lugs extending radially outward from a proximal end portion of the loading unit, and wherein relative rotation between the loading unit and the elongated shaft mechanically and electrically couples the loading unit and the elongated shaft together.

26. The surgical stapling apparatus of claim 21, wherein the housing electrical connector assembly includes a connector housing and a plurality of spring contacts supported in the connector housing.

27. The surgical stapling apparatus of claim 26, wherein adjacent springs of the plurality of spring contacts are longitudinally spaced apart from one another.

28. The surgical stapling apparatus of claim 27, wherein the loading unit electrical connector assembly includes an electrical coupler having a plurality of longitudinally spaced apart contact rings that is configured to contact the plurality of spring contacts for electrically coupling the housing electrical connector assembly to the loading unit electrical connector assembly.

29. The surgical stapling apparatus of claim 28, wherein the elongated shaft further includes a proximal seal supported proximal to the housing electrical connector assembly and the electrical coupler of the loading unit electrical connector assembly supports a distal seal, wherein the proximal seal and the distal seal cooperate to seal the housing electrical connector assembly and the loading unit electrical connector assembly within the elongated shaft when the housing electrical connector assembly and the loading unit electrical connector assembly are electrically coupled together.

30. The surgical stapling apparatus of claim 29, wherein the plurality of spring contacts of the housing electrical connector assembly is connected to a first flex cable that extends proximally to the housing assembly, and the plurality of longitudinally spaced apart contact rings of the loading unit electrical connector assembly is connected to a second flex cable that extends distally to the at least one sensor.

31. A surgical stapling apparatus, comprising:
a housing assembly;
an elongated shaft extending from the housing assembly and supporting a housing electrical connector assembly therein; and
a loading unit selectively electrically connectable to the elongated shaft by relative translating and rotating movement between the loading unit and the elongated shaft, the loading unit supporting a loading unit electrical connector assembly, the housing electrical connector assembly positioned to receive the loading unit electrical connector assembly to cause the elongated shaft and the loading unit to electrically couple together in response to the translating and rotating movement.

32. The surgical stapling apparatus of claim 31, wherein the loading unit includes at least one sensor that is disposed in electrical communication with the loading unit electrical connector assembly.

33. The surgical stapling apparatus of claim 32, wherein the loading unit electrical connector assembly is disposed on a proximal end portion of the loading unit and the at least one sensor is longitudinally spaced from the loading unit electrical connector assembly.

34. The surgical stapling apparatus of claim 33, wherein the elongated shaft supports a firing rod and defines lug slots therein for receiving lugs extending radially outward from the proximal end portion of the loading unit, and wherein relative rotation between the loading unit and the elongated shaft mechanically and electrically couples the loading unit and the elongated shaft together.

35. The surgical stapling apparatus of claim 34, wherein the housing electrical connector assembly includes a connector housing and a peripheral seal secured onto the connector housing.

36. The surgical stapling apparatus of claim 35, wherein the housing electrical connector assembly further includes a plurality of spring contacts supported in the connector housing, and wherein adjacent springs of the plurality of spring contacts are longitudinally spaced apart from one another.

37. The surgical stapling apparatus of claim 36, wherein the loading unit electrical connector assembly includes an electrical coupler having a plurality of longitudinally spaced apart contact rings that is configured to contact the plurality of spring contacts for electrically coupling the housing electrical connector assembly to the loading unit electrical connector assembly.

38. The surgical stapling apparatus of claim 37, wherein the elongated shaft further includes a proximal seal supported about the firing rod proximal to the housing electrical connector assembly and the electrical coupler of the loading unit electrical connector assembly supports a distal seal, wherein the proximal seal and the distal seal cooperate to seal the housing electrical connector assembly and the loading unit electrical connector assembly within an elongated shaft assembly when the housing electrical connector assembly and the loading unit electrical connector assembly are electrically coupled together.

39. The surgical stapling apparatus of claim 38, wherein the plurality of spring contacts of the housing electrical connector assembly is connected to a first flex cable and the plurality of longitudinally spaced apart contact rings of the loading unit electrical connector assembly is connected to a second flex cable.

40. A surgical stapling apparatus, comprising:
an elongated shaft supporting a housing electrical connector assembly; and
a loading unit selectively attachable to the elongated shaft and extending distally to an end effector supporting at least one sensor, the loading unit supporting a loading unit electrical connector assembly on a proximal end portion of loading unit, the loading unit electrical connector assembly electrically coupled to the at least one sensor and positioned to contact the housing electrical connector assembly when the loading unit electrical connector assembly is received within the elongated shaft to electrically couple the at least one sensor to the housing electrical connector assembly.
